(19) United States
(12) Patent Application Publication
(10) Pub. No.: US 2022/0015929 A1
(43) Pub. Date: Jan. 20, 2022

(54) ADJUSTABLE PYRAMID ADAPTER FOR PROSTHETIC DEVICE

(71) Applicant: OTTO BOCK HEALTHCARE LP, Austin, TX (US)

(72) Inventors: Brandon ANDERSON, West Jordan, UT (US); Vaughn Roy ANDERSON, Highland, UT (US)

(73) Assignee: OTTO BOCK HEALTHCARE LP, Austin, TX (US)

(21) Appl. No.: 17/359,281

(22) Filed: Jun. 25, 2021

Related U.S. Application Data
(60) Provisional application No. 63/044,720, filed on Jun. 26, 2020.

(51) Int. Cl. A61F 2/66; A61F 2/70

(52) U.S. Cl. CPC A61F 2/66; A61F 2002/502; A61F 2/70

(57) ABSTRACT

A prosthetic device and related methods includes an elongate support member having a first surface, and an adapter mounted to the support member. The adapter includes a base portion, a movable member, a connector portion, and an adjustment member. The adjustment member is operable to move the movable member relative to the base portion to change an effective length of an interface between the adapter and the first surface of the support member. The connector portion extends from the base portion and is configured to releasably secure the prosthetic foot to a prosthesis.

### TECHNICAL FIELD

The present disclosure relates generally to prosthetic devices, and more particularly relates to adapters and connectors for prosthetic devices, and related methods of manufacturing and use of such adapters and connectors.

### BACKGROUND

Prosthetic feet generally include spring-like members which are flexible and resilient. To mimic, as closely as possible, the function of a human foot the members must deflect under the user's weight. Unfortunately, most existing prosthetic feet are not nearly as adaptable to varying uses and conditions as the human foot. Prosthetic feet provide trade-offs between flexibility and durability. In general, stiff prosthetic foot components are stronger than more flexible components and strong components are stiffer than more flexible components. During strenuous, high load activities, a stiff and strong prosthetic foot components provide more optimum support and strength an amputee requires while more flexible components provide more bending function and comfort during less demanding activities.

Moreover, matching the stiffness of the foot to the desires of an amputee is challenging. Depending on the activity an amputee is performing (i.e., walking vs. running) an improved prosthetic foot will provide some user adjustability to provide optimum performance during different activities and/or some adjustment so a prosthetic foot can be fine-tuned to a particular user's preference.

Such flexibility and the ability to deflect often require the spring members forming the foot to be structurally weak, or more flexible. On the other hand, it is desirable to make the members as strong or stiff as possible from a structural and durability standpoint. Thus, there may be a trade-off between obtaining a sufficient cushion or feel, with spring members that are weak or flexible and over-deflect, and obtaining a solid and durable structural foot, with stiffer members.

The stiffness of prosthetic feet typically varies according to the intended use. Feet intended for everyday use are typically too fragile for athletic use. Multiple-use feet have been designed which are capable of many different uses, but without being particularly well-suited for any specialized use. In addition, users may have different weights. Thus, prosthetic feet may require a high degree of custom design or be particularly tailored to the individual user. However, it is desirable from a cost and manufacturing standpoint to create a foot that is usable by individuals of varying weight, strength, walking style, and preferred activities.

The connection point between the prosthetic adapter and the upper foot plate is often a challenge for prosthetic foot designers. This is typically the location that experiences the most extreme stress in a prosthetic foot, therefore is important to optimize the connection to maximize both foot durability and flexibility. Over time, prosthetic foot designers have improved the connection between prosthetic adapters and the remaining components of a foot. Prosthetic adapters are typically made of metal and the upper foot plate is typically made of a fiber reinforced composite laminate.

A first generation of a prosthetic adapter interface was flat and square or rectangular with four bolt holes at the corners of the interface: two at the anterior end and two at the posterior end of the adapter. This resulted in high stresses in the anterior bolts, high contact stresses in the upper foot plate at the anterior edge of the pyramid, and poor roll-over characteristics for the foot.

Typically, the practitioner would select the most suitable prosthetic foot for the “most used” circumstances of the user. If the user wanted to engage in higher level, less common activities, the practitioner would prescribe an additional prosthetic foot that would be suitable this other level of activity. It was not uncommon for an amputee to have several different types of prosthetic feet that can be swapped out, depending on the current activity of the user. Also, pediatric users of prosthetic feet typically grow out of their prosthetic feet on a regular basis. This problem has typically been solved by simply replacing the foot on a regular basis, or fitting the patient with a foot that is not optimal, but one that the user can grow into.

For these and other reasons, there is a need to provide improved prosthetic feet and respective components that are functional and/or adaptable for a variety of activities and user needs.

### SUMMARY

In order to satisfy numerous patients and their specific circumstances, a foot spring with a smoothly increasing stiffness may be advantageous for the foot and its performance. One aspect of the present disclosure relates to a prosthetic foot having an adjustable length pyramid elongate composite spring member connector, which can be adjusted to modify a stiffness of the prosthetic device. One objective of the present disclosure is to provide one or more adjustment features associated with the pyramid connector that modify characteristic of the foot over a wide variety of user activities and other parameters.

Another aspect of the present disclosure relates to a pyramid adapter assembly that is used in conjunction with a prosthetic foot. The pyramid adapter includes a mechanism wherein the user or practitioner can lengthen or shorten an end of the adapter (e.g., anterior end) and thus change the stiffness of the prosthetic foot. As the adapter is lengthened, the foot spring is essentially shortened and the foot becomes stiffer. As the adapter is shortened the foot becomes softer. With this simple adjustment the prosthetic foot can be optimized for the user and/or the user's short term requirements (i.e., the activity level, weight, and the intended use of the foot).

Another aspect of the present disclosure relates to a prosthetic foot that is adaptable to, for example, different activities, overall amputee activity level, amputee weights, and personal preference. The present disclosure also relates to an adapter for use with a prosthetic device, such as a prosthetic foot, that allows the practitioner to adjust the stiffness of the prosthetic foot to correspond to the user's physical requirements, lifestyle, and activity level. This not only is advantageous for the user's current circumstances, but can be easily adjusted as the user experiences physical changes in anatomy and/or lifestyle.

In one embodiment, a prosthetic foot includes an elongate support member comprising fiber reinforced material and having a first surface, and an adapter mounted to the support member. The adapter includes a base portion, a movable member, and an adjustment member. The adjustment member is operable to move the movable member relative to the base portion to change an effective length of an interface between the adapter and the first surface of the support member.

The adapter may also include a connector portion, which extends from the base portion and is configured to releasably secure the prosthetic foot to a prosthesis. One of the base portion and the movable portion may have a cavity, and the other of the base portion and the movable member may be positioned at least partially within the cavity. The adjustment member may include a threaded shaft, and rotating the threaded shaft may move the adjustment member relative to the base portion. The adjustment member may include a motor operable to move the movable member relative to the base portion. The prosthetic foot may also include at least one sensor configured to detect at least one condition associated with use of the prosthetic foot, and generate a sensor signal used to control the adjustment member. The prosthetic foot may include a controller and at least one sensor, and the controller and at least one sensor may be operable to automatically control the adjustment member in response to at least one sensed condition associated with the prosthetic foot. The connector portion may have a central axis, and the movable member may be movable in a direction parallel with the central axis. The connector portion may have a central axis, and the movable member may be movable in a direction perpendicular to the central axis.

Another embodiment is directed to a prosthetic foot that includes a foot plate spring member, a top spring member secured to the foot plate spring member and having a first surface, and an adapter. The adapter includes a base portion having a contact surface arranged to contact the first surface of the top spring member, a movable member, an adjustment member operable to move the movable member relative to the base portion to change a stiffness of the prosthetic foot, and a connector portion extending from the base portion and configured to releasably secure the prosthetic foot to a prosthesis.

One of the base portion and the movable portion may have a cavity, and the other of the base portion and the movable member may be positioned at least partially within the cavity. The adjustment member may move the movable member to change an effective length of an interface between the adapter and the top spring member. The adjustment member may be configured to be automatically operated in response to a detected condition of use of the prosthetic foot. The adjustment member may be configured to be manually operated.

A further embodiment relates to a method of adjusting a stiffness of a prosthetic device. The method includes providing a prosthetic device comprising an elongate composite spring member having a first surface, and an adapter. The adapter includes a base portion, a movable member, an adjustment member, and a connector portion, wherein the connector portion extends from the base portion and is configured to releasably secure the prosthetic device to a prosthesis. The method also includes operating the adjustment member to move the movable member relative to the base portion to adjust an effective length of an interface between the adapter and the first surface of the spring member to adjust the stiffness of the prosthetic device.

The adjustment member may include at least one threaded shaft, and operating the adjustment member may include rotating the at least one threaded shaft. Operating the adjustment member may include manually rotating a portion of the adjustment member. Operating the adjustment member may include automatically operating the adjustment member in response to a detected condition of use associated with the prosthetic device. Operating the adjustment member may move the movable member in a vertical direction. The first surface faces vertically upward, and operating the adjustment member may move the movable member in a generally horizontal direction. Operating the adjustment member may include operating an electric motor mounted to the prosthetic device.

Another embodiment is directed to a prosthetic foot that includes a top foot plate spring member, and an adapter in contact with the top foot plate spring member. The adapter includes a movable portion and a base portion. The base portion is fixed relative to the top foot plate spring member, and the movable portion is movable relative to the base portion to change a distance between lines of contact between the adapter and the top foot plate spring member during a gait cycle. These lines of contact may be referred to as contact points. These contact lines appear as points in a two dimensional image such as the figures of the present application (e.g., a side view or a cross section of a foot or prosthetic device). The lines of contact typically extend in the width direction (medial/lateral) and depending on the orientation the adapter mounted on a prosthetic foot. These lines may be described as proximal most and distal most, or a combination of proximal and posterior most, or distal and anterior most, or any combination of anterior, posterior, proximal and distal.

The adapter may include a movable portion and a base portion, the base portion may be fixed relative to the top foot plate spring member, and the adjustable portion may be operable to move the movable portion relative the base portion to change the distance between the posterior most and anterior most contact points between the adapter and the top foot plate spring member. Operating the adjustable portion may change a stiffness property of the prosthetic foot. The foot stiffness property may be determined by measuring a vertical deflection of the prosthetic foot at a point of load application when applying a load of, for example, 750 N with the prosthetic foot oriented, for example, at 20 degrees toe down or 15 degrees toe up, where 0 degrees is an orientation of the prosthetic foot when unloaded and resting on a horizontal surface with a shim under a heel of the prosthetic foot that is equal to a thickness of a specified heel height of the prosthetic foot. The lines of contact may extend in a medial/lateral direction.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a cross-sectional side view of a portion of a prosthetic foot in accordance with the present disclosure.

FIG. 2 is a cross-sectional side view of the portion of prosthetic foot shown in FIG. 1.

FIG. 3 is a front view of the portion of prosthetic foot shown in FIG. 1.

FIG. 3A is a front view of another example prosthetic foot in accordance with the present disclosure.

FIG. 4 is a side view of the portion of prosthetic foot shown in FIG. 1 in a first adjusted position.

FIG. 5 is a side view of the portion of prosthetic foot shown in FIG. 1 in a second adjusted position.

FIG. 6 is a cross-sectional side view of a portion of another prosthetic foot in accordance with the present disclosure.

FIG. 7 is a close up view of a portion of the prosthetic foot shown in FIG. 6.

FIG. 8 is a cross-sectional side view of a portion of a prosthetic foot in accordance with the present disclosure.

FIG. 9 is a cross-sectional side view of a portion of a prosthetic foot in accordance with the present disclosure.

FIG. 10 is a cross-sectional side view of a portion of a prosthetic foot in accordance with the present disclosure.

FIG. 11 is a cross-sectional side view of a portion of another prosthetic foot in accordance with the present disclosure.

FIG. 12 is a close up view of a portion of the prosthetic foot shown in FIG. 11.

FIG. 13 is a cross-sectional side view of a portion of a prosthetic foot in accordance with the present disclosure.

FIG. 14 is a block diagram schematically representing an example adjustment manager in accordance with the present disclosure.

FIG. 15 is a schematic diagram of an example system in accordance with the present disclosure.

FIG. 16 is a flow diagram showing steps of an example method in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure is generally directed to prosthetic devices, and more particularly relates to adaptors (e.g., pyramid adapters) for prosthetic devices, and related methods for using such adaptors.

In order to satisfy numerous patients and their specific circumstances, a prosthetic foot with an adjustable stiffness is a desirable aspect of the foot and its performance. The present disclosure provides a pyramid adapter (also referred to as a pyramid connector, adapter, or connector) with an adjustment feature that provides the stiffness adjustment for the prosthetic foot. The pyramid adapter may have an adjustable length, wherein the longer the effective length of the pyramid adapter, the stiffer the prosthetic foot becomes. The effective length may include, but not be limited to, a length of an interface in an anterior/posterior direction between the pyramid adapter and a top surface of a spring member of the prosthetic device to which the pyramid adapter contacts and is mounted. In some examples, the effective length may be measured or determined at least in part based on a connection point between the adapter and the spring to which the adapter is mounted. The connection point may be defined by, for example, a mounting bolt that provides releasable attachment of the adapter to the spring member. The effectively length typically is measured or determined when the prosthetic device is in a rest position or state, prior to application of a user's weight force or use of the prosthetic device by a user.

Lower limb prosthetic components may benefit from adjustability and are typically connected using an industry standard prosthetic pyramid connection. A prosthetic pyramid connection typically consists of a male pyramid connector/adapter and a complementary female pyramid connector/adapter connected to each other. The combination of the male and female adapters may provide for angular adjustment between two prosthetic components. The male portion may include two primary features: a pyramid protrusion and a contoured (e.g., spherical) surface. The pyramid protrusion may have four planar surfaces that are oriented in posterior, anterior, medial, and lateral directions. These surfaces may be angled with respect to a pyramid axis, wherein the pyramid axis extends along a longitudinal axis of the shin or thigh of the limb. The pyramid surfaces typically are angled in the range of about 10 degrees to about 30 degrees, and more particularly about 15 degrees. Due to the angles of the four pyramid surfaces, the protrusion necks down in a distal direction. The necked down end transitions to the contoured (e.g., spherical) surface. The contoured portion may be part of a separate base component with the pyramid protrusion fixedly and rigidly attached to the base component. Alternatively, the base component may be integrated to the spherical feature where the two features are combined into a single monolithic block of material.

The pyramid protrusion may be threaded into the base component with the threads glued or otherwise fixed to prevent unthreading. Alternate methods of fixedly attaching a pyramid protrusion to a base component including a spherical surface are possible such as, for example, creating a stud on the narrow end of the pyramid protrusion and molding the stud into a fiber reinforced moldable base material, or by deforming the stud such that the stud creates a strong interference fit between the a pyramid protrusion and the base component. A male pyramid adapter may be monolithic meaning it is formed or composed of a single, continuous material without joints or seams. However, as discussed herein, the male pyramid adapter may comprise a plurality of components, such as one or more components that adjust an effective length of the pyramid adapter.

A female pyramid adapter may include a predominantly hollow cylinder with a spherical surface formed on one end and four threaded fasteners. The inner surface of the cylinder may not be round or cylindrical as recesses are commonly formed on this surface to allow increased articulation of the male protrusion within the cylinder while adjusting the angle between the components. The spherical surfaces of both the male and female components have a near identical spherical radius to allow mating with each other. Fasteners (e.g., two or more fasteners) may be threaded into the cylinder at an angle relative to the cylinder axis (e.g., 15 degree angle), and the fasteners may engage one or more of the four planar surfaces of the male pyramid protrusion to releasably secure the position of the pyramid connection. By adjusting the depth of the fasteners in the female component, the angle between the male and female pyramid components can be changed and the angle between two prosthetic components can be adjusted. A female pyramid component may be referred to as a pyramid receiver. A female pyramid adapter may be monolithic. The threaded fasteners typically are separate components in a monolithic female pyramid adapter.

The male and female pyramid adapter may each have fastening provisions to be attached to adjacent components, such as holes for attaching the pyramid adapter to components of a prosthetic foot or prosthetic knee using, for example, fasteners (e.g., bolts or rivets), a clamp, or a bonding surface for bonding the adapter to an adjacent component such as prosthetic pylon, which may be, for example, a composite or metal tube. A male or female pyramid adapter component may be machined or formed directly onto a prosthetic device, for example, a prosthetic knee. For the purposes of this disclosure, a pyramid adapter may be either the male or female component of a pyramid connection and include either a pyramid protrusion and a spherical mating surface in the case of a male pyramid adapter or a spherical mating surface with multiple (e.g., four) threaded fasteners to engage and lock a pyramid protrusion in the case of a female adapter. A pyramid adapter may be fabricated separately from other components and include design features allowing the adapter to be attached to other prosthetic components in addition to connecting the complementary opposite component of a pyramid connection.

The present disclosure relates to prosthetic feet that typically include a rigid pyramid adapter. A rigid pyramid adapter does not allow movement within the adapter. Some pyramid adapters include one or more axes of rotation within the adapter that allow the pyramid protrusion to rotate relative to the base of the adapter. Rotation about the axes may be controlled or limited by a hydraulic circuit, a bumper, or a mechanism that allows the heel height of the foot to be adjusted. The devices of the present disclosure are generally directed to pyramid adapters that are rigid and are intended to provide angular adjustment between the male and female adapter components of the pyramid connection, but not movement or articulation within either the male or female adapter components. Pyramid adapters that provide movement and adjustment within the adapter are typically more expensive to manufacture and may suffer from wear at surfaces associated with the axes that allow movement and any other component that restricts, controls, or prevents movement. This may result in reduced reliability. Prosthetic feet with such adapters may be referred to as hydraulic ankles, hydraulic feet, single axis feet, or adjustable heel height feet. A consequence of the high loads pyramid connections are subjected to and the desire to minimize the weight of all prosthetic components, is that a rigid pyramid adapter may exhibit a small amount of elastic deformation under the high forces imposed during a walking gait cycle, thus resulting in a small amount of angular change (e.g., less than two degrees) between any two surfaces within a pyramid connection.

The present disclosure, among other things, provides a prosthetic foot with a mechanical mechanism to alter the inherent stiffness of a prosthetic foot. The prosthetic foot may include an adjustment feature to alter the fulcrum point of the pyramid adapter, thereby altering the location where the adapter contracts the prosthetic spring member. By lengthening the adapter fulcrum point, the lever arm acting on foot spring member and the stressed induced in the spring member are reduced and the stiffness of the foot increases. By shortening the fulcrum point, the inherent stiffness of the foot is decreased. This simple adjustment mechanism allows the prothesis to optimize the prosthetic foot for one or more of the size and weight of the user and the activity level of the user. In embodiments in which the adjustment mechanism is operable by the user, the user can make stiffness changes to optimize the stiffness for a specific activity.

In one embodiment, the device incorporates a screw adjusted mechanism that extends an effective length of an interface between the adapter and the top spring member of the foot. The mechanism may include an insert in the pyramid adapter. By turning the adjustment screw the pyramid adapter increases or decreases in length to change the fulcrum point where the pyramid adapter contacts the spring member, causing the prosthetic foot to be more or less stiff in the dorsiflexion direction.

In another embodiment, the device incorporates a lockable slide insert that is contained within the pyramid adapter. By turning the adjustment screw the pyramid adapter can be extended in length to change the fulcrum point where the pyramid adapter contacts the spring member, thus yielding a more or less stiff prosthetic foot in the dorsiflexion.

In yet another embodiment, the pyramid adapter has a multi-piece construction and is configured such that a portion of the adapter can be adjusted growing the length of the adapter. Extending the length of the adapter effectively changes the stiffness of the prosthetic foot in dorsiflexion.

In yet another embodiment, adjustment to the pyramid adapter is accomplished by an electrical motor. The motor may be controllable remotely (e.g., via Bluetooth communication with a cell phone, computer, smart watch, or other transmitting device). In one embodiment, the adjustments are implemented passively (i.e., set one time before the desired activity) or dynamically (e.g., adjustments are made during the desired activity based on sensor feedback).

In yet another embodiment, the adjustment to the pyramid adapter is accomplished by an electric motor in combination with a microprocessor and sensors to detect a usage mode, a walking speed, a stress or deflection in a foot or other prosthetic component. The microprocessor may be operable to determine the usage mode and adjust the pyramid adaptor to provide the optimum stiffness.

In yet another embodiment, the moving components are held it place with a spring force (e.g., a preloaded Belleville washer) to minimize or eliminate rattle/noise.

These and other objects, features and advantages of the present invention become more apparent from a consideration of the following detailed description of disclosed example embodiments of the invention and the accompanying drawings.

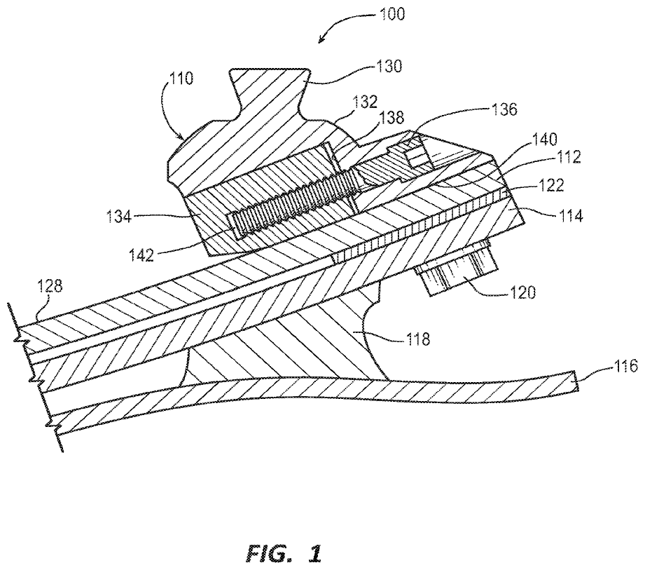

Referring now to FIGS. 1-5, an example prosthetic device 100 in the form of a prosthetic foot is shown and described. Prosthetic device 100 includes an adaptor 110, a top spring member 112, a second spring member 114, a foot plate spring member 116, and a heel bumper 118. The adaptor is secured to the spring members 112, 114 with a mounting bolt 120. A spacer 122 may be positioned between the spring members 112, 114 in the area of the mounting bolt 120. The adaptor 110 may be positioned on a top surface 128 of the top spring member 112. That portion of the adaptor 110 that is facing and in contact with the top surface 128 when the prosthetic device 100 is in a rest position may be referred to as and/or define an interface between the adaptor 110 and the top spring member 112. In at least some arrangements, the mounting bolt 120 is positioned posterior or rearward of a connector feature of the adaptor 110. The mounting bolt 120 may be connected to a rearward or posterior portion of the adaptor 110, such as a portion of the adaptor 110 that is positioned furthest from a toe end of the prosthetic device 100.

The arrangement of spring members 112, 114, 116 of the prosthetic device 100 may vary in different embodiments. Other embodiments may include a single top spring member 112 that is connected directly to the foot plate spring member 116 without an intervening second spring member 114. The heel bumper 118 may be interposed between and in contact with both the top spring member 112 and the foot plate spring member 116. In other embodiments, the prosthetic device is free of a heel bumper 118. Another embodiment may include only a heel bumper or cushion without a distal foot plate spring member 116. Other embodiments may include a single spring member, such as a spring member that extends in a rearward direction from the adaptor and then curves forward to the toe end of the prosthetic device. Regardless of the number of spring members, their orientation, size or shape, the principals disclosed herein related to the adaptor and its adjustable features (e.g., a length adjustment feature) may provide benefits and advantages for any given prosthetic foot or prosthetic device generally that includes an adaptor.

FIG. 1 shows the adaptor 110 having a connector 130, a base 132, and a movable member 134, and an adjustment member 136. The base 132 includes a cavity 138 and a bottom surface 140. The movable member 134, also referred to as a fulcrum member, an adjustable member, a movable fulcrum member, a stiffness adjustment member, or stiffness member, is positioned in the cavity 138. The adjustment member 136 extends through the base 132 and into contact with the movable member 134. The movable member 134 includes a threaded bore 142 that is threadably engaged by the adjustment member 136.

The adjustment member 136 may include a bolt having threads at one end and a head at an opposite end. The head is configured to be engaged with a tool that provides rotation of the adjustment member 136. Other types of adjustment members are contemplated, including other types of fasteners, brackets, and assemblies.

The movable member 134 may have a block-type construction. The movable member 134 may slide relative to the base 132 in a forward or anterior direction and a rearward or posterior direction. The movable member 134 may slide along the top surface 128 of the top spring member 112 while the base 132 remains in a fixed position by connection via the mounting bolt 120. Operating the adjustment member 136 moves the movable member 134 relative to the base 132 and the top spring member 112.

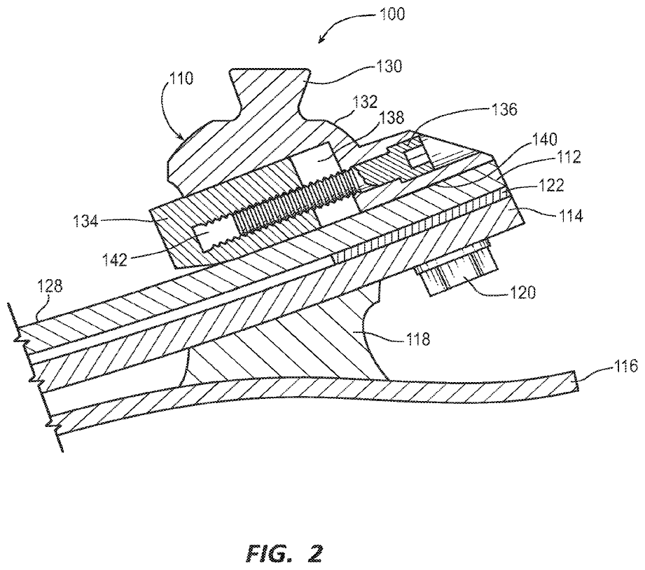

FIG. 1 shows the movable member 134 in a fully retracted or near fully retracted position relative to the base 132 so as to be in the furthermost rearward position. FIG. 2 shows the movable member 134 advanced in the forward direction upon operation of the adjustment member 136. The extent to which the movable member 134 may be moved in the forward direction relative to the base 132 may be depend on a variety of factors including, for example, the length of the threaded bore 142, the length of the adjustment member 136 extending into the movable member 134, the size and shape of the movable member 134, and the size of cavity 138.

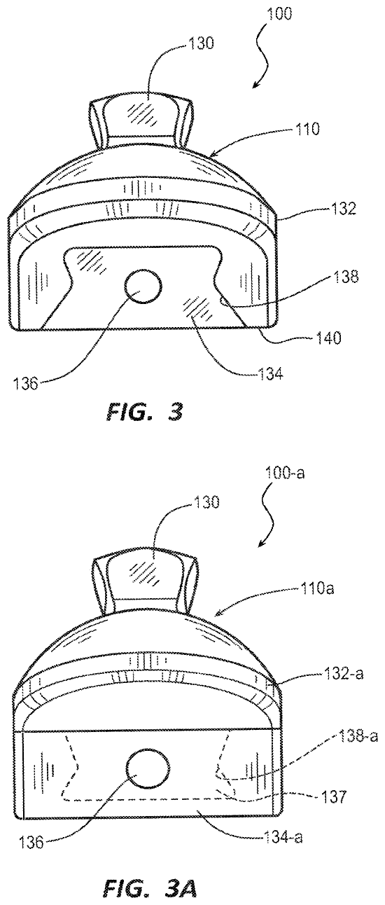

FIG. 3 shows a front view of the adaptor 110. The movable member 134 may have a dovetail-shaped perimeter that mates with a corresponding dovetail-shaped cavity 138 in the base 132. The mating shape of the cavity 138 and the movable member 134 may help retain the movable member 134 within the base 132 and provide a track along which the movable member 134 advances and is retracted upon operation of the adjustment member 136. Many different cross-sectional shapes may be possible for the movable member 134 and cavity 138.

In at least some examples, the movable member 134 may be designed to have a maximum surface area along the bottom surface 140 of the adaptor 110 thereby providing increased stability, force transfer, etc., between the movable member 134 and the top spring member 112. The shape of the bottom surface 140 may be varied to provide other characteristics and properties for the prosthetic device. Some example bottom surface constructions, shapes, and considerations are disclosed in U.S. Patent Application No. 62/984,170, filed on 2 Mar. 2020, which is incorporated herein by reference.

FIG. 3A shows a front view of an alternative adaptor 110-a design wherein the movable member 134-a has a dovetail-shaped cavity 138-a sized to receive a dovetail-shaped protrusion 137 of the base 132-a. The cavity 138-a may be defined by three interior walls of the movable member 134-a, including a front wall that defines an anterior surface of the movable member 134-a. The movable member 134-a may be adjusted in an anterior/posterior direction relative to the base 132-a using the adjustment member 136. The adjustment member 136 may be operable from a front or anterior side of the adaptor 110-a, although it may be possible to operate the adjustment member 136 from a rear or posterior side of the base 132-a in at least some embodiments. The adaptor 110-a may generally provide for a movable member 134-a having a cavity 138-a, and a portion 137 of the base 132-a is positioned in and provides a position connection with the movable portion 134-a while permitting relative movement between the base 132-a and the movable member 134-a.

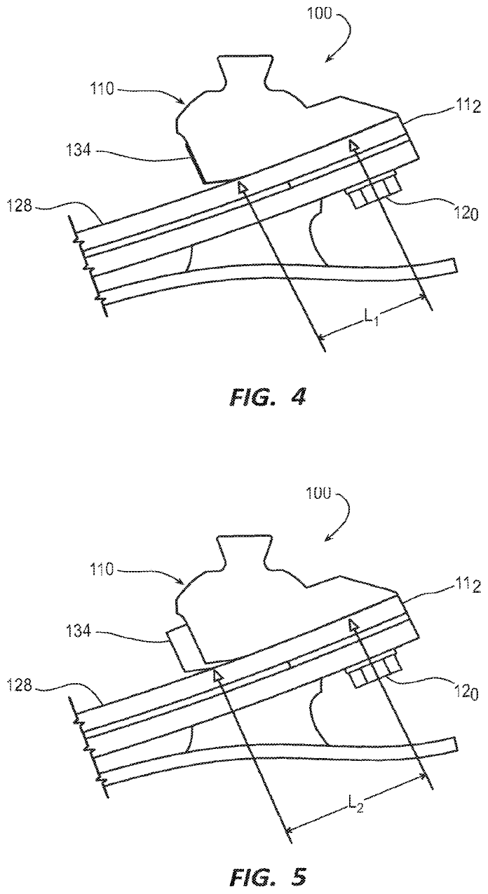

Referring to FIGS. 4 and 5, a length of a moment arm defined by the adaptor 110 is described. FIG. 4 shows a length L₁ of the moment arm being defined between a central axis of the mounting bolt 120 and the forwardmost point of contact between the base 132 and the top surface 128 of the top spring member 112. This forward most (also referred to as anterior most depending on the orientation of the adapter) contact point between the adaptor 110 and the top spring member 112 may be referred to as a fulcrum point. FIG. 4 shows the movable member 134 in the fully retracted or rearward most position, such that the fulcrum point is adjusted to a rearward or posterior most position relative to the top spring member 112.

FIG. 5 shows the movable member 134 advanced in a forward direction thereby increasing the length L₂ of the moment arm. The increased length L₂ effectively increases the stiffness and strength of the prosthetic foot 100 by positioning the fulcrum point further towards the anterior and/or distal end of the top spring member 112. Advancing the movable member 134 further in the forward or anterior direction may further increase the effective length L of the moment arm, thereby providing additional increased stiffness for the prosthetic foot 100.

As discussed above, the ability to change the effective length of the moment arm provided by the adaptor 110 may provide the ability to customize the stiffness of the prosthetic foot 100 for a given activity of the user or other parameter such as, for example, the height, weight, or stability needs of the user. In at least some examples, the adaptor 110 may be adjustable to change the length of the moment arm during the process of fitting and/or setting up the prosthetic device for a particular user. The prosthesis may take into consideration the user's intended activity level as well as other considerations related to the user such as height and weight and set the stiffness level of the prosthetic foot in a way that accommodates the broadest range of use for the user. In other examples, the user or prosthesis may operate the adjustment member 136 to adjust the effective moment arm length L on an as-needed basis, such as just before or after undertaking particular activities such as exercise, ascending or descending stairs or ramps, or extended periods of standing in one place, thereby providing a desired stiffness characteristic for the prosthetic device 100 that meets that particular need for a shorter period of time.

Generally, the foot stiffness can be determined using a variety of different measurements and/or methods. For example, the foot stiffness may be determined by measuring a vertical deflection of the foot at a point of load application when applying a load of 750 N with the foot oriented 20 degrees toe down or 15 degrees toe up, where 0 degrees is an orientation of the foot when unloaded and resting on a horizontal surface with a shim under a heel of the foot that is equal to a thickness of a specified heel height of the foot.

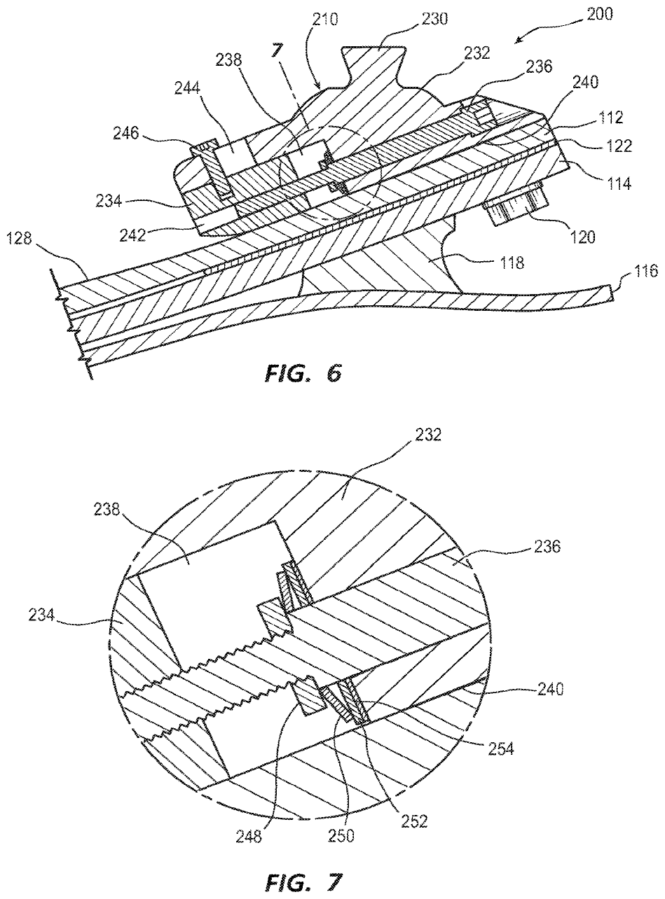

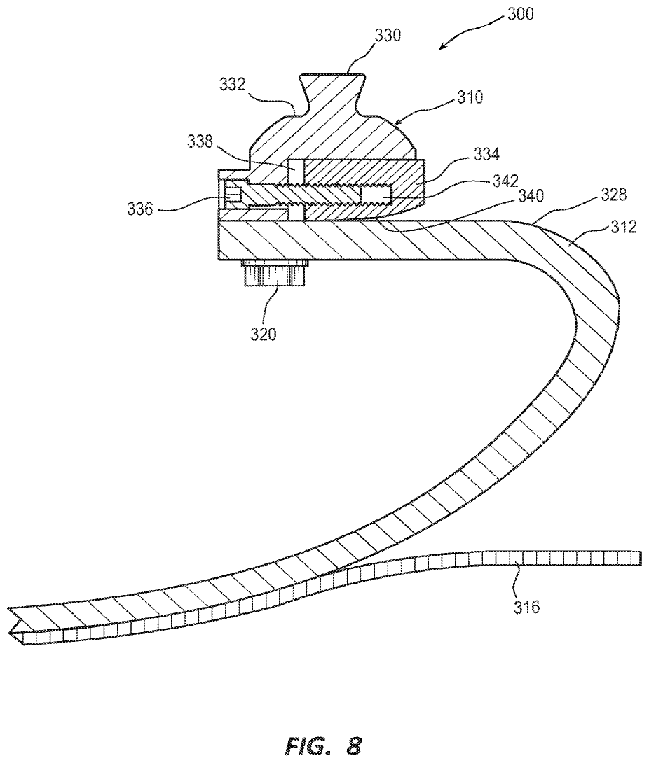

FIGS. 6 and 7 show another example prosthetic device 200 having additional features associated with an adjustment member 236. The prosthetic device 200 is shown as a prosthetic foot, but features disclosed herein may be applicable to other types of prosthetic devices. The prosthetic device 200 includes an adaptor 210 that is mounted to the top surface 128 of a top spring member 112. The adaptor 210 includes a connector 230, a base 232, an movable member 234, and the adjustment member 236. The base 232 includes a cavity 238, a bottom surface 240, a set screw slot 244, and a set screw 246 extending through the set screw slot 244 and into contact with the movable member 234. The set screw 246 may be operable to fix a position of the movable member 234 relative to the base 232.

The adjustment member 236 may include a nut 248, a conical washer 250, a low friction washer 252, and a standard washer 254. The nut 248 and washers 250, 252, 254 may be positioned within the cavity 238 of the base 232. The nut 248 and washers 250, 252, 254 may help retain the adjustment member 236 in a given rotated position to prevent inadvertent advancement or retracting of the movable member 234 relative to the base 232. The nut 248 and washers 250, 252, 254 may help prevent noise during use of the prosthetic device 200. In at least some embodiments, the adjustment member 236 with nut 248 and washers 250, 252, 254 may maintain an adjusted position of the movable member 234 without the use of set screw 246. The set screw 246 with set screw slot 244 may be used in other embodiments, such as the embodiment of adaptor 110 described above, to help hold the movable member 234 at a fixed position relative to the base 232.

The base 232 may have a longer nose portion extending in an anterior direction as compared to the adaptor 110 described above. The extended nose length may provide advantages related to, for example, providing space for an elongated set screw slot 244, providing improved support for the movable member 234 along a length of travel relative to the base 232, and/or accommodating additional components within the cavity 238 such as, for example, one or more of the nut 248 and washers 250, 252, 254 while still providing the same amount of adjustment length for the movable member 234 relative to the base 232.

The conical washer 250 may provide a tension or locking function between the nut 248 and the base 232. The low friction washer 252 may provide a reduced friction interface between the base 232 and the conical washer 250. An additional low friction washer 252 may be positioned between the conical washer 250 and the nut 248. In at least some examples, the adjustment member 236 does not include a separate standard washer 254 at the interface between the low friction washer 252 and the cavity 238 of the base 232. Many other types of washers, nuts, and interface features of the adjustment member 236 may be provided to create the same or similar functions related to fixing a location of the movable member 234 relative to the base 232.

FIG. 8 shows another example prosthetic device 300 that includes an adaptor 310 mounted to a spring member 312 that extends in a rearward direction from the adaptor 310 before curving downward and forward towards the toe end of the prosthetic device 300. The prosthetic device 300 may be a prosthetic foot or other type of prosthetic device or component thereof. A heel spring member 316 (also referred to as a foot plate spring member 316) may be mounted to the spring member 312 and extend in a rearward or posterior direction from a toe end of the spring member 312 to define a heel surface for the prosthetic device 300. In at least some examples, the prosthetic device 300 is operable without the heel spring member 316.

The adaptor 310 is secured to the spring member 312 with a mounting bolt 320. The adaptor 310 is positioned in contact with an upper or top surface 328 of the spring member 312. The adaptor 310 includes a connector 330, a base 332, a movable member 334, and an adjustment member 336. The base 332 includes a cavity 338 and a bottom surface 340 that is arranged facing and in contact with the top surface 328 of the spring member 312. Operating the adjustment member 336 moves the movable member 134 in a rearward direction relative to the base 332 to increase the stiffness of the prosthetic device 300 as a result of changing the fulcrum point between the movable member 134 and the spring member 312 further rearward. Retracting the movable member 134 in a forward or anterior direction relative to the base 132 reduces the stiffness of the prosthetic device 300. Thus, operating the adjustment member 336 may change or adjust the stiffness of the prosthetic device 300, and may do so by moving the movable member 334 (and the fulcrum point) in a reverse direction compared to the embodiments shown in FIGS. 1-7.

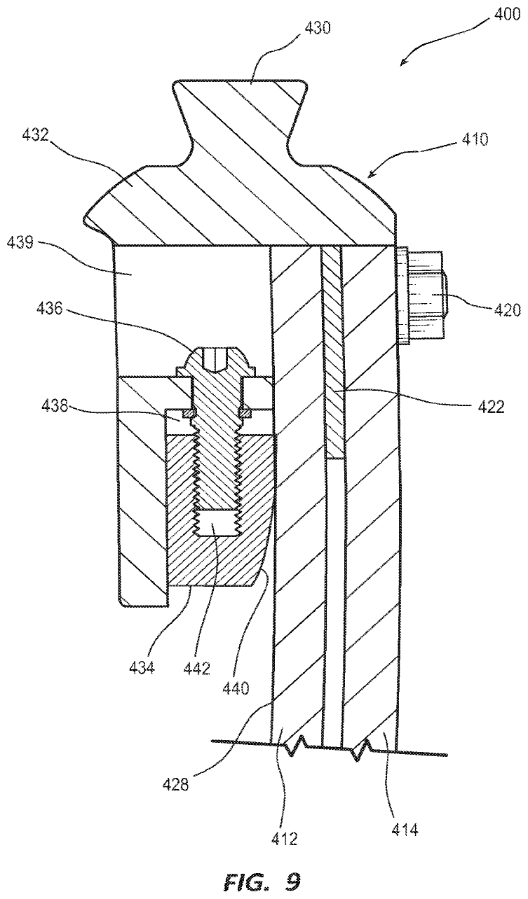

FIG. 9 shows another example prosthetic device 400 having an adaptor 410 mounted to a top spring member 412 and second spring member 414. The prosthetic device 400 may be a prosthetic foot or other prosthetic device or component thereof. The spring members 412, 414 are arranged in a vertical direction with a spacer 422 positioned therebetween. Only the proximal ends of the spring members 412, 414 are shown. The remaining distal or anterior ends of the spring members 412, 414 may curve in a forward or anterior direction, thereby defining a toe end of the prosthetic device 400.

The adaptor 410 includes a connector 430, a base 432, an movable member 434, and an adjustment member 436. The base 432 includes at least one cavity 438 and a bottom surface 440 that faces and contacts a top surface 428 of the top spring member 412.

The movable member 434 is positioned at least partially within the cavity 438. Operating the adjustment member 436 moves the movable member 434 relative to the base 432. Typically, the movable member 434 slides along the top surface 428 of the top spring member 412. The movable member 434 may move in a generally vertical direction, such as in a direction towards a distal lower end of the prosthetic device or in an opposite direction towards the proximal upper end of the prosthetic device 400.

The adjustment member 436 may be accessible through a second cavity 439 defined in the base 432. In at least some examples, the adjustment member 436 is arranged in a vertical direction, or at least in a direction parallel with top surface 428. Adjusting the position of the movable member 434 relative to the base 432 may adjust an effective moment arm length between the mounting bolt 420 and the distal contact point between the movable member 434 and the top surface 428. Operating the adjustment member 436 to move the movable member 434 may change a stiffness characteristic of the prosthetic device 400. In at least one example, adjusting the movable member 434 in a downward or distal direction relative to the base 432 may increase the stiffness of the prosthetic device 400. Retracting the movable member 434 in a proximal direction that may be in a vertically upward direction relative to the base 432 may reduce a stiffness property of the prosthetic device 400.

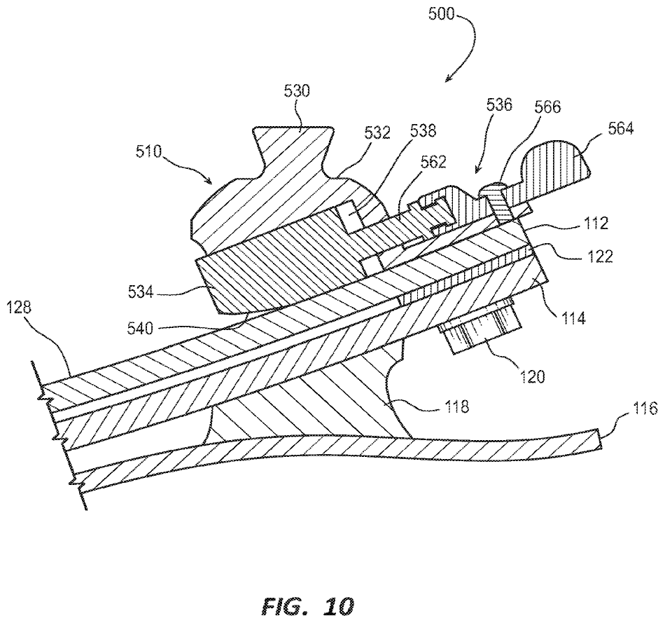

FIG. 10 shows another example prosthetic device 500 having an adaptor 510 mounted to a top spring member 112 and second spring member 114 with a mounting bolt 120. The prosthetic device 500 may be a prosthetic foot or other prosthetic device or component thereof. A spacer 122 may be positioned between the top and second spring members 112, 114. A foot plate spring member 116 may be connected to the spring members 112, 114 as described above. A heel bumper 118 may be positioned between the second spring member 114 and foot plate spring member 116.

The adaptor 510 may include a connector 530, a base 532, a movable member 534, and an adjustment assembly 536. The base 532 may include a cavity 538 and a bottom surface 540 that faces and contact the top surface 128 of the top spring member 112. The movable member 534 is positioned at least partially within the cavity 538.

The adjustment assembly 536 may include a cam assembly that operates to move the movable member 534 in a forward or anterior direction relative to the base 132 and in a rearward or posterior direction relative to the base 532. The cam assembly may include a shaft 562 connected to the movable member 534, and a cam member 564 connected to the shaft and supported by a pivot post 566. Moving the cam 564 about the post 566 may move the movable member 534 in the forward and rearward directions within the cavity 538 relative to the base 532. The cam 564 may have a variety of shapes, sizes, and configurations. In at least some examples, the cam 564 includes a lever or other actuating surface that a user can apply force to thereby operating the adjustment assembly 536. In at least one example, the cam 564 is movable back and forth within a generally horizontal plane, or a plane that is relatively parallel with the top surface 528. In other examples, the cam 564 may be operable to move in a vertically up and down direction about the post 566 oriented in a different orientation such as within a relatively horizontal plane. Other types of cam features may be used to provide a similar function by converting a rotational motion of the cam member into a translational movement of the movable member 534 in a forward and rearward direction relative to the base 532. Other types of adjustment assemblies may include bearing surfaces, ball and socket joints, cam surfaces (e.g., variable radius surfaces), and the like to provide the desired adjustment. In at least some examples, the cam may have a plurality of preset adjusted positions that relate to certain adjusted positions of the movable member 534 relative to the base 532.

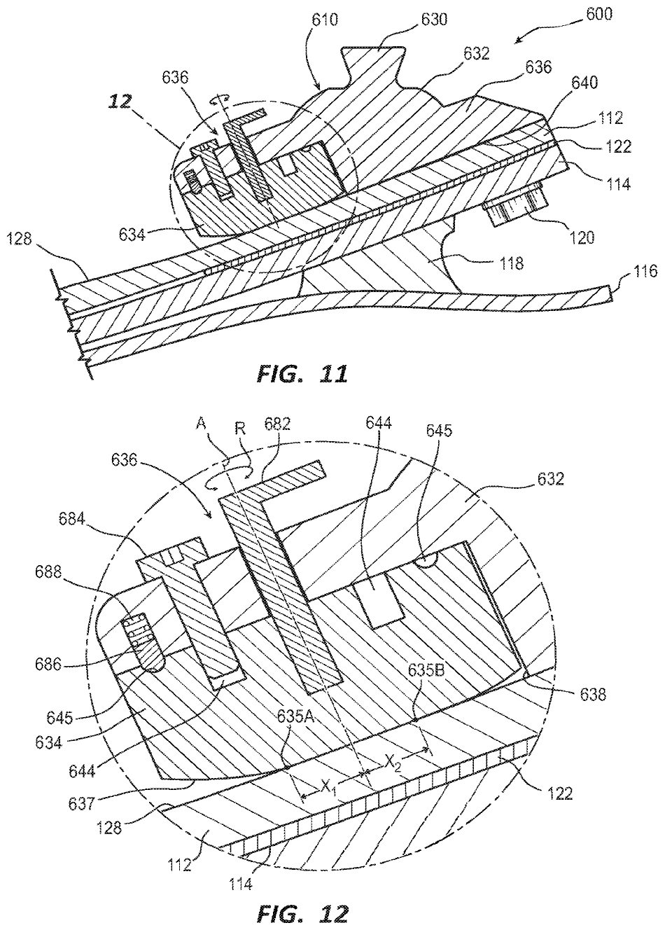

FIGS. 11 and 12 show another example prosthetic device 600 having an adaptor 610 mounted to a top spring member 112 and second spring member 114 with a mounting bolt 120. The prosthetic device 600 may be in the form of, for example, a prosthetic foot. A spacer 122 may be positioned between the top and second spring members 112, 114. A foot plate spring member 116 may be connected to the spring members 112, 114 as described above. A heel bumper 118 may be positioned between the second spring member 114 and foot plate spring member 116.

The adaptor 610 may include a connector 630, a base 632, a movable member 634, and an adjustment assembly 636. The base 632 may include a cavity 638 and a bottom surface 640 that faces and contacts the top surface 128 of the top spring member 112. The movable member 634 is positioned at least partially within the cavity 638.

The movable member 634 is rotatable within the cavity 638 about a rotation axis A. The movable member 634 is asymmetrical about the rotation axis A. As a result, a surface 637 of the movable member 634 that faces the top surface 128 of the top spring member 112 has different distances X₁, X₂ from a contact point 635A, 635B and the rotation axis A. The movable member 634 may include a plurality of contact points in addition to the contact points 635A, 635B at various on the surface 637 of the movable member 634.

The movable member 634 may be rotated into different rotated positions about the rotation axis A to provide a variable fulcrum point in a forward direction (also referred to as an anterior or distal direction) between the movable member and the top surface 128 of the top spring member 112. Changing the fulcrum point between the adaptor 610 and the top spring member 112 may influence the stiffness and other properties of the prosthetic device 600 as discussed with reference to other embodiments disclosed herein.

The movable member 634 may include a plurality of set screw bores 644 to receive the set screw 684 to retain the movable member 634 in a desired rotated position about the axis A. In other embodiments, the set screw 684 contacts an outer surface of the movable member 634 rather than being inserted into a set screw bore, and contact between the set screw 684 provides a force that resists rotation of the movable member 634.

The movable member 634 may also include a plurality of detent recesses 645 sized and arranged to receive one or more of the detent 686. The detent 686 may be biased by one or more springs 688 that help retain the detent 686 in the detent recess 645 to help hold the movable member 634 in a predetermined rotated position, such as holding the movable member 634 in a desired rotated position until the set screw 684 is actuated to engage the movable member 634. Other types of fasteners and adjustable retaining features may be used in addition to or in place of the set screw 684 and detent 686 to help hold the movable member 634 in a desired rotated position with an associated fulcrum point and stiffness adjustment for the prosthetic device 600.

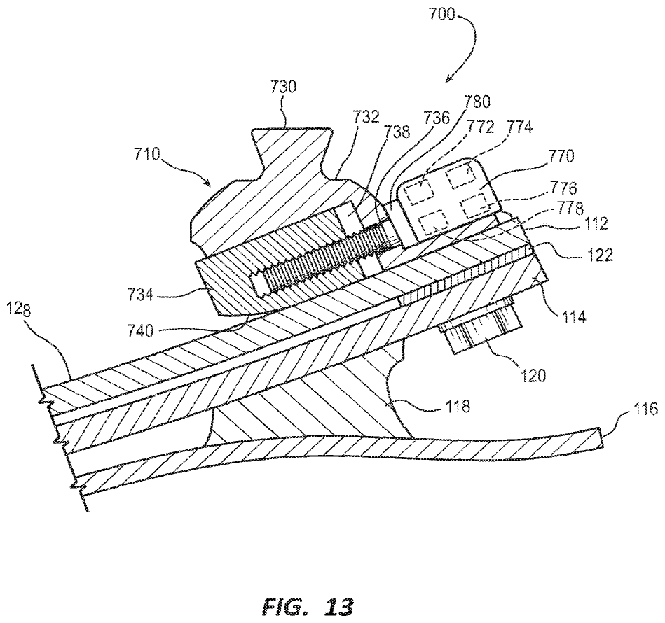

FIG. 13 shows an example prosthetic device 700 having an adaptor 710 mounted to a top spring member 112 and a second spring member 114 via a mounting bolt 120. The prosthetic device 700 may be a prosthetic foot or other prosthetic device or component thereof. A spacer 122 may be positioned between the spring members 112, 114 in the area of the mounting bolt 120. A separate foot plate spring member 116 may be connected to the spring members 112, 114. A heel bumper 118 may be positioned between the second spring member 114 and the foot plate spring member 116. The adaptor 110 is mounted to the top spring member 112 along a top surface 128.

The adaptor 710 includes a connector 730, a base 732, a movable member 734, and an adjustment member 736. The base 732 includes a cavity 738 and a bottom surface 740 that faces and contact the top surface 128 of the top spring member 112. The movable member 734 is positioned within the cavity 738 and movable in the forward or anterior direction and rearward or posterior direction relative to the base 732.

The adjustment member 736 may include one or more electronic features or components that are operable to assist with moving the movable member 734 relative to the base 732. For example, the adjustment member 736 may include a motor 770 that is operable to rotate the adjustment member 736 to move the movable member 734. The adaptor 110 may also include a processor 772, sensor 774, a switch 776, a transceiver 778, and a power source 780. The processor 772 may be programmed to operate the motor 770 in response to one or more command signals. For example, the processor 772 may be connected wirelessly to a remote computing device, such as a user's handheld mobile device. The command may be to operate the motor 770 to change a stiffness of a prosthetic device 700 based on an adjusted position of the movable member 734 for a particular activity that the user is going to undertake. Alternatively, the processor 772 may receive a plurality of sensor signals from the sensor 774 and determine that the user has undertaken a certain activity and then adjust the position of the movable member 734 to provide a change in stiffness for that particular activity. Some example sensors include accelerometers, absolute angle sensors, force sensors, velocity sensors, position encoders, a stepper motor, strain gauges, piezoelectric sensors and/or signal generators, and the like. Although the sensor 774 are shown positioned at a particular location on the prosthetic device 700, other embodiments may include one or more of the sensors 774 positioned at other locations on the prosthetic device 700 or at other locations on the user such as, for example, at different positions proximal of the prosthetic device 700.

The switch 776 may be a manual switch, such as an on/off switch for the processors 772 and/or motor 770. The switch 776 may be operable electronically. The transceiver 778 may be used to transmit and receive signals to and from the processor 772, sensor 774, switch 776, power 780, or other components of the prosthetic device 700. The transceiver 778 may be connected wirelessly with a remote computing system such as, for example, a mobile handheld computing device.

The power 780 may include, for example, a battery power source. The power 780 may be a rechargeable power source. The power 780 may provide a source of power for operation of any of the electronic components of the prosthetic device 700 including, for example, the motor 770, processor 772, sensor 774, switch 776, and transceiver 778.

The prosthetic device 700 may further include memory to store one or more software or firmware programs, sensor data, or instructions that are sent or received as part of communications with a remote computing device.

The ability to provide electronic control of the positioning of movable member 734 may provide a variety of advantages as compared to embodiments that provide only manual operation of the movable member position. Automatic foot performance adjustment based on terrain or activity detection may allow a foot to automatically adjust to the user's immediate environment. Manual adjustment of a foot to optimize, for example, a short stair ascent or descent is likely to be a nuisance whereas automatic adjustment requires no user input. However, a simple and easy to execute manual adjustment may be highly advantageous, for example, in longer term exposures to different environments such as indoor verses outdoor activities.

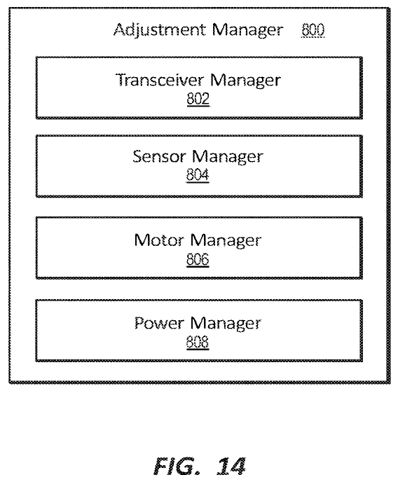

FIG. 14 shows a block diagram of an adjustment manager 800. The adjustment manager 800 may include one or more processors, memory, and/or one or more storage devices. The adjustment manager 800 may include transceiver manager 802, sensor manager 804, motor manager 806, and power manager 808. The managers shown in FIG. 14 may each be in communication with each other. The managers shown in FIG. 14 may perform at least one of the operations described herein in conjunction with one or more controllers, transmitters, receivers, or other features of the adjustment member, adjustable pyramid adapter, and/or prosthetic feet disclosed herein. The adjustment manager 800 may be one example of a manager or module that is operated by a prosthetic device or adapter disclosed herein, such as prosthetic device 600 and/or adapter 610 described with reference to FIGS. 11-12.

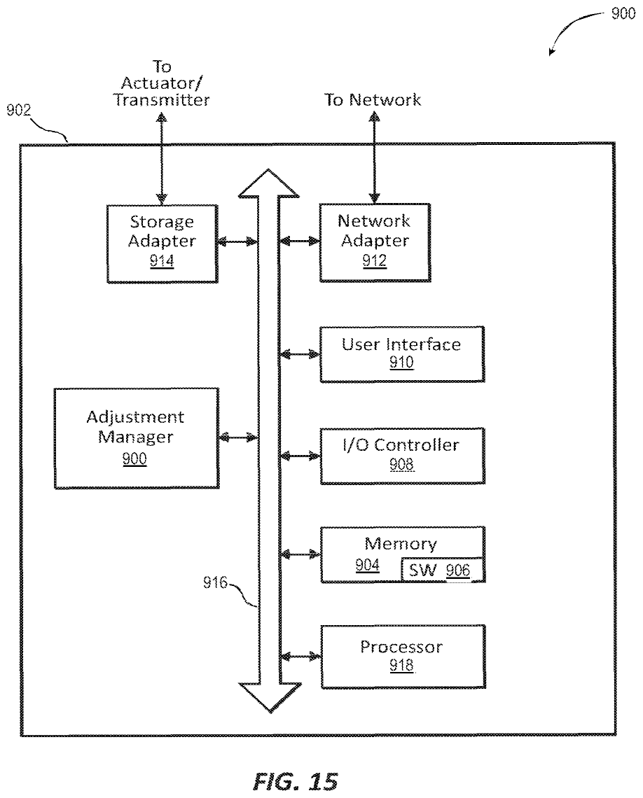

The transceiver manager 802 may operate to control sending and receiving of data, instructions, or the like. The transceiver manager 802 may provide wireless communication such as using one or more of the wireless communication systems or networks described below with reference to FIG. 15. The transceiver manager 802 may be in communication with one or more processors, memory and/or storage devices associated with the adjustment manager 800.

The sensor manager 804 may include one or more sensors and/or provide communication between one or more sensors and one or more processors, memory and/or storage devices. The sensor manager 804 may facilitate communication of signals received from one or more sensors. Sensor manager 804 may facilitate communication to one or more sensors to provide a desired function.

The motor manager 806 may operate to control one or more motors or other power generating devices for operation of the movable member of the adjustable adaptors disclosed herein. The motor manager 806 may provider on/off control of a motor. Motor manager 806 may provide adjustments in, for example, the amount of power used in the prosthetic device and/or the amount of power available for use, and other operational aspects related to the motor or other devices to assist with moving the movable member. The motor manager 806 may monitor the motor and collect data including, for example, useful life, maintenance schedule, and the like.

Power manager 808 may control distribution of power within the adjustable adaptor. For example, power manager 808 may control storage of power, monitor power capacity, and/or control distribution of power amongst various electronic components of the adjustable adaptor.

FIG. 15 shows a control system 900 for use with a prosthetic device. System 900 may include an apparatus 902, which may be an example of the electronic components and/or the adaptor 710 of prosthetic device 700 shown in FIG. 13.

Apparatus 902 may include components for wired or wireless control of the adapters and/or prosthetic devices disclosure herein and related communications including components for transmitting communications and components for receiving communications. For example, apparatus 902 may communicate bi-directionally with one or more actuators and/or transmitters, and a network, such as a data network. These bi-directional communications may be direct (apparatus 902 communicating directly with an actuator/transmitter or network, for example) and/or indirect (apparatus 902 communicating indirectly with another device through a server, for example).

Apparatus 902 may also include a processor module 918, memory 904 (including software/firmware code (SW) 906), an input/output controller module 908, a user interface 910, a network adapter 912, and a storage adapter 914. The software/firmware code 906 may be one example of a software application executing on apparatus 902. The network adapter 912 may communicate bi-directionally, via one or more wired links and/or wireless links, with one or more networks and/or client devices. In some embodiments, network adapter 912 may provide a direct connection to a client device via a direct network link to the Internet via a POP (point of presence). In some embodiments, network adapter 912 of apparatus 902 may provide a connection using wireless techniques, including digital cellular telephone connection, Cellular Digital Packet Data (CDPD) connection, digital satellite data connection, and/or another connection. The apparatus 902 may include adjustment manager 800, which may perform the functions described above for the adjustment manager of FIG. 14.

The signals associated with system 900 may include wireless communication signals such as radio frequency, electromagnetics, local area network (LAN), wide area network (WAN), virtual private network (VPN), wireless network (using 802.11, for example), cellular network (using 3G and/or LTE, for example), and/or other signals. The network adapter 912 may enable one or more of WWAN (GSM, CDMA, and WCDMA), WLAN (including BLUETOOTH® and Wi-Fi), WMAN (WiMAX) for mobile communications, antennas for Wireless Personal Area Network (WPAN) applications (including RFID and UWB), or any combination thereof. Such wireless communications signals may be used with any of the devices and systems disclosed herein, such as the prosthetic device 600 disclosed herein.

One or more buses 916 may allow data communication between one or more elements of apparatus 902 such as processor module 918, memory 904, I/O controller module 908, user interface 910, network adapter 912, and storage adapter 914, or any combination thereof.

The memory 904 may include random access memory (RAM), read only memory (ROM), flash memory, and/or other types. The memory 904 may store computer-readable, computer-executable software/firmware code 906 including instructions that, when executed, cause the processor module 918 to perform various functions described in this disclosure. Alternatively, the software/firmware code 906 may not be directly executable by the processor module 918 but may cause a computer (when compiled and executed, for example) to perform functions described herein. Alternatively, the computer-readable, computer-executable software/firmware code 906 may not be directly executable by the processor module 918, but may be configured to cause a computer, when compiled and executed, to perform functions described herein. The processor module 918 may include an intelligent hardware device, for example, a central processing unit (CPU), a microcontroller, an application-specific integrated circuit (ASIC), field programmable gate array (FPGA), or any combination thereof.

In some embodiments, the memory 904 may contain, among other things, the Basic Input-Output system (BIOS) which may control basic hardware and/or software operation such as the interaction with peripheral components or devices. For example, at least a portion of the adjustment manager 800 to implement the present systems and methods may be stored within the system memory 904. Applications resident with system 900 are generally stored on and accessed via a non-transitory computer readable medium, such as a hard disk drive or other storage medium. Additionally, applications can be in the form of electronic signals modulated in accordance with the application and data communication technology when accessed via a network interface such as network adapter 912.

Many other devices and/or subsystems may be connected to and/or included as one or more elements of system 900 (for example, a personal computing device, mobile computing device, smart phone, server, internet-connected device, cell radio module, or any combination thereof). In some embodiments, all of the elements shown in FIG. 15 need not be present to practice the present systems and methods. The devices and subsystems can be interconnected in different ways from that shown in FIG. 15. In some embodiments, an aspect of some operation of a system, such as that shown in FIG. 15, may be readily known in the art and are not discussed in detail in this application. Code to implement the present disclosure can be stored in a non-transitory computer-readable medium such as one or more of system memory 904 or other memory. The operating system provided on I/O controller module 908 may be a mobile device operation system, a desktop/laptop operating system, or another known operating system.

The I/O controller module 908 may operate in conjunction with network adapter 912 and/or storage adapter 914. The network adapter 912 may enable apparatus 902 with the ability to communicate with devices such as prosthetic device 700 of FIG. 13, and/or other devices over a communication network. Network adapter 912 may provide wired and/or wireless network connections. In some cases, network adapter 912 may include an Ethernet adapter or Fibre Channel adapter. Storage adapter 914 may enable apparatus 902 to access one or more data storage devices. The one or more data storage devices may include two or more data tiers each. The storage adapter 914 may include one or more of an Ethernet adapter, a Fibre Channel adapter, Fibre Channel Protocol (FCP) adapter, a SCSI adapter, and iSCSI protocol adapter.

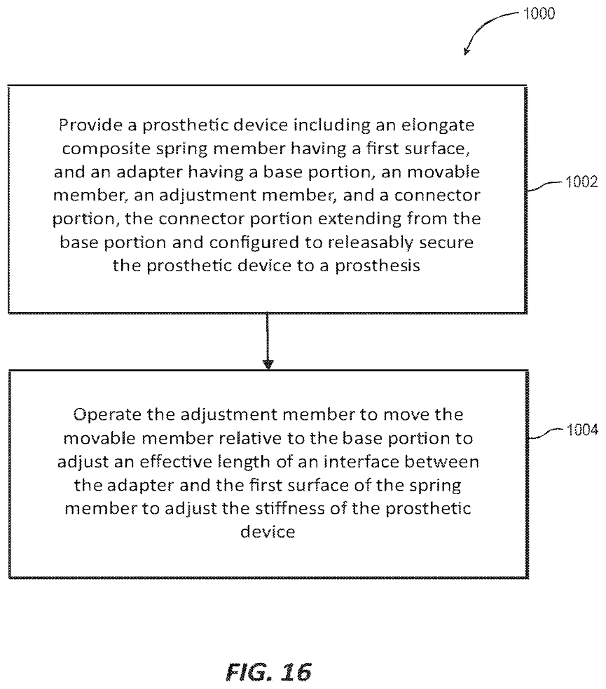

FIG. 16 is a flow diagram illustrating an example method 1000 of adjusting a stiffness of a prosthetic device, such as a prosthetic foot. The method 1000 may represent one or more steps applicable to operation of any one of the prosthetic devices and/or adaptors described above with reference to FIGS. 1-15. For example, the steps of method 1000 may reflect operation of prosthetic device 100 and adaptor 110 described above with reference to FIGS. 1-5. While several method steps associated with method 1000 are shown in FIG. 14, other variations of related methods of adjusting a stiffness of a prosthetic device and/or use of an adjustable pyramid adaptor in accordance with the present disclosure may include more or fewer steps than those shown in FIG. 14.

The method 1000, at block 1002, includes providing a prosthetic device having an elongate composite spring member with a first surface, and an adaptor having a base portion, a movable member, an adjustment member, and a connector portion. The connector portion extends from the base portion and is configured to releasably secure the prosthetic device to a prosthesis. Block 1004 of method 1000 includes operating the adjustment member to move the movable member relative to the base portion to adjust an effective length of an interface between the adaptor and the first surface of the spring member to adjust the stiffness of the prosthetic device.

The method 1000 may additionally or alternatively include providing the adjustment member with at least one threaded shaft, and operating the adjustment member may include rotating the threaded shaft. Operating the adjustment member may include manually rotating a portion of the adjustment member. Operating the adjustment member may include automatically operating the adjustment member in response to a detected condition and/or use associated with the prosthetic device. The prosthetic device may be a prosthetic foot, and the spring member may include at least a top spring member extending from a proximal end to a distal end (e.g., heel end to toe end) of the prosthetic foot. Operating the adjustment member may move the movable member in a vertical direction. When the first surface facing vertically upward, operating the adjustment member may move the movable member in a generally horizontal direction. Operating the adjustment member may include operating an electric motor mounted to the prosthetic foot. Operating the adjustment member may occur in response to one or more sensor signals that detect a predetermined use of the prosthetic device and/or physical property in association with use of the device.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

1. A prosthetic foot, comprising:
an elongate support member comprising fiber reinforced material and having a first surface;
an adapter comprising:
a base portion;
a movable member;
an adjustment member operable to move the movable member relative to the base portion to change an effective length of an interface between the adapter and the first surface of the support member.

2. The prosthetic foot of claim 1, wherein one of the base portion and the movable portion has a cavity, and the other of the base portion and the movable member is positioned at least partially within the cavity.

3. The prosthetic foot of claim 1, wherein the movable member extends in an anterior direction from the base portion.

4. The prosthetic foot of claim 1, wherein the adjustment member includes a threaded shaft, and rotating the threaded shaft moves the adjustment member relative to the base portion.

5. The prosthetic foot of claim 1, wherein the adjustment member includes a motor operable to move the movable member relative to the base portion.

6. The prosthetic foot of claim 1, further comprising at least one sensor configured to detect at least one condition associated with use of the prosthetic foot, and generate a sensor signal used to control the adjustment member.

7. The prosthetic foot of claim 1, further comprising a controller and at least one sensor, the controller and at least one sensor operable to automatically control the adjustment member in response to at least one sensed condition associated with the prosthetic foot.

8. The prosthetic foot of claim 1, wherein the adapter further comprises a connector portion extending from the base portion and configured to releasably secure the prosthetic foot to a prosthesis.

9. The prosthetic foot of claim 8, wherein the connector portion has a central axis, and the movable member is movable in a direction parallel with the central axis.

10. The prosthetic foot of claim 8, wherein the connector portion has a central axis, and the movable member is movable in a direction perpendicular to the central axis.

11. A prosthetic foot, comprising:
a foot plate spring member;
a top spring member secured to the foot plate spring member and having a first surface;
an adapter comprising:
a base portion having a contact surface arranged to contact the first surface of the top spring member;
a movable member having a contact surface arranged to contact the first surface of the top spring member;
an adjustment member operable to move the movable member relative to the base portion to change a stiffness of the prosthetic foot;
a connector portion extending from the base portion and configured to releasably secure the prosthetic foot to a prosthesis.

12. The prosthetic foot of claim 11, wherein the adjustment member moves the movable member to change an effective length of an interface between the adapter and the top spring member.

13. The prosthetic foot of claim 11, wherein the adjustment member is configured to be automatically operated in response to a detected condition of use of the prosthetic foot.

14. The prosthetic foot of claim 11, wherein the adjustment member is configured to be manually operated.

15-22. (canceled)

23. A prosthetic foot comprising;
a top foot plate spring member;
an adapter in contact with the top foot plate spring member, the adapter including a movable portion and a base portion, the base portion being fixed relative to the top foot plate spring member, and the movable portion being movable relative to the base portion to change a distance between lines of contact between the adapter and the top foot plate spring member during a gait cycle.

24. The prosthetic foot of claim 23, wherein operating the adjustable portion changes a stiffness of the prosthetic foot.

25. The prosthetic foot of claim 24, wherein the foot stiffness is determined by measuring a vertical deflection of the prosthetic foot at a point of load application when applying a load of 750 N with the prosthetic foot oriented 20 degrees toe down or 15 degrees toe up, where 0 degrees is an orientation of the prosthetic foot when unloaded and resting on a horizontal surface with a shim under a heel of the prosthetic foot that is equal to a thickness of a specified heel height of the prosthetic foot.

26. The prosthetic foot of claim 23, wherein the lines of contact extend in a medial/lateral direction.
